(19) United States
(12) Patent Application Publication
(10) Pub. No.: US 2017/0285153 A1
(43) Pub. Date: Oct. 5, 2017

(54) ULTRASONIC DEVICE, ULTRASONIC MEASUREMENT APPARATUS, AND ULTRASONIC IMAGE DISPLAY

(71) Applicant: SEIKO EPSON CORPORATION, Tokyo (JP)

(72) Inventor: Ryuichi SATO, Shiojiri-shi (JP)

(73) Assignee: SEIKO EPSON CORPORATION, Tokyo (JP)

(21) Appl. No.: 15/453,235

(22) Filed: Mar. 8, 2017

(30) Foreign Application Priority Data
Mar. 29, 2016 (JP) 2016-065204

(51) Int. Cl. G01S 7/52; A61B 8/08; G01S 15/89; A61B 8/00

(52) U.S. Cl. CPC G01S 7/52004; A61B 8/4444; A61B 8/461; A61B 8/0841; A61B 8/4483; G01S 15/8918

(57) ABSTRACT

An ultrasonic device includes a plurality of ultrasonic element arrays each of which is provided with a plurality of ultrasonic elements arranged in a first direction, in which the plurality of ultrasonic element arrays are disposed at positions which do not interfere with each other in the first direction, and are disposed at positions which are deviated relative to each other in a second direction intersecting the first direction.

### BACKGROUND

### 1. Technical Field

The present invention relates to an ultrasonic device, an ultrasonic measurement apparatus, and an ultrasonic image display.

### 2. Related Art

In the related art, there are an ultrasonic probe including a one-dimensional array vibrator (one-dimensional array) in which a plurality of vibrating elements (ultrasonic elements) transmitting and receiving ultrasonic waves are disposed in one direction, and an ultrasonic diagnosis apparatus (ultrasonic measurement apparatus) including the ultrasonic probe (for example, refer to JP-A-2012-105751).

The ultrasonic measurement apparatus disclosed in JP-A-2012-105751 includes a convex type or a sector type ultrasonic element array radially transmitting ultrasonic waves from each ultrasonic element in a predetermined scanning plane, or a linear type ultrasonic element array linearly transmitting ultrasonic waves from each ultrasonic element. The ultrasonic measurement apparatus configured in the above-described way can acquire a tomographic image of a measurement target in the scanning plane.

Meanwhile, when ultrasonic measurement is performed by using an ultrasonic probe including a one-dimensional array, ultrasonic tomographic images corresponding to a plurality of measurement positions can be acquired by inclining the ultrasonic probe or moving the ultrasonic probe along a normal direction to a scanning plane.

However, it is not easy to perform ultrasonic measurement with high accuracy while reducing a distance between measurement positions.

For example, in a mechanical ultrasonic measurement apparatus which includes a driving mechanism moving an ultrasonic probe along a normal direction to a scanning plane or changing an inclination of the scanning plane of the ultrasonic probe, and is configured to be able to change a measurement position, there is concern that the apparatus may be complex and become large in size. In such a mechanical ultrasonic measurement apparatus, there is concern that the scanning accuracy may be reduced due to vibration or the like emitted from the driving mechanism.

On the other hand, a position of the scanning plane or an inclination of the scanning plane may be manually changed. However, in this case, there is concern that the scanning accuracy may be reduced due to vibration in the same manner as in the mechanical ultrasonic measurement apparatus. Since a manual operation is performed, it is not easy to quantitatively specify a position of the scanning plane, that is, a measurement position.

There may be a configuration in which a plurality of ultrasonic arrays are disposed in a normal direction to the scanning plane. In this case, however, a distance between scanning planes, that is, an interval between measurement positions is restricted depending on an outer dimension of the ultrasonic array in the normal direction, and thus there is a limitation in reducing a measurement position interval.

### SUMMARY

An advantage of some aspects of the invention is to provide an ultrasonic device, an ultrasonic measurement apparatus, and an ultrasonic image display, capable of performing highly accurate ultrasonic measurement while reducing a measurement position interval.

An ultrasonic device according to an application example includes a plurality of ultrasonic element arrays each of which is provided with a plurality of ultrasonic elements arranged in a first direction, in which the plurality of ultrasonic element arrays are disposed at positions which do not interfere with each other in the first direction, and are disposed at positions which are deviated relative to each other in a second direction intersecting the first direction.

In the plurality of ultrasonic element arrays in this application example, ultrasonic elements are arranged in the first direction. The plurality of ultrasonic element arrays are disposed at positions which do not interfere with each other in the first direction. In other words, a certain ultrasonic element array and another ultrasonic element array adjacent to the ultrasonic element array in the first direction are disposed to be adjacent to or separate from each other in the first direction (positions in the first direction do not overlap each other). The plurality of respective ultrasonic element arrays are disposed at positions which are deviated relative to each other in the second direction.

Here, the ultrasonic element array is configured as, for example, a so-called one-dimensional array which transmits and receives ultrasonic waves along a scanning plane including a central line which passes through the center of the ultrasonic element array in the second direction and is parallel to the first direction. Since the ultrasonic element array also has a predetermined width dimension in the second direction intersecting the scanning plane, in a case where the ultrasonic element arrays are disposed at the same position in the first direction and are arranged in the second direction, an interval between the scanning planes of the respective ultrasonic element arrays are not smaller than a width dimension of the ultrasonic element array, and thus a measurement position interval in the second direction is increased. In contrast, in the application example, since the ultrasonic element arrays are disposed at positions which do not overlap each other in the first direction, the ultrasonic element arrays adjacent to each other in the second direction intersecting the scanning plane can be disposed at any positions. Therefore, a distance between the scanning planes in the second direction, that is, a measurement position interval can be set regardless of an outer dimension of the ultrasonic element array in the second direction, and can thus be made smaller than the outer dimension of the ultrasonic element array.

Since the ultrasonic element arrays are disposed in advance with a predetermined positional relationship, compared with a case in which the ultrasonic element array is moved ore rotated using a driving mechanism as described above, there is no influence of vibration or the like of the driving mechanism, and highly accurate ultrasonic measurement can be performed. Compared with a case of the manual operation, similarly, there is no influence of vibration or the like, and a distance to a measurement position can be more accurately specified.

As mentioned above, according to the application example, it is possible to provide an ultrasonic device which can perform highly accurate ultrasonic measurement while reducing a measurement position interval.

In the ultrasonic device according to the application example, it is preferable that a deviation amount in the second direction in ultrasonic element arrays adjacent to each other in the first direction among the plurality of ultrasonic element arrays is smaller than a width in the second direction of each of the ultrasonic element arrays adjacent to each other.

In the application example with this configuration, a deviation amount (for example, a deviation amount of the central line) in the second direction in ultrasonic element arrays adjacent to each other in the first direction among the plurality of ultrasonic element arrays is smaller than a width of each of the ultrasonic element arrays adjacent to each other. In other words, a certain ultrasonic element array and another ultrasonic element array adjacent to the ultrasonic element array in the first direction are disposed to partially overlap each other in the second direction. In this configuration, a distance between the scanning planes in the second direction, that is, a measurement position interval can be made smaller than an outer dimension of the ultrasonic element array in the second direction.

In the ultrasonic device according to the application example, it is preferable that each of the ultrasonic element arrays has a measurement region based on a set transmission angle range of an ultrasonic wave in a plane including the first direction, and the measurement regions corresponding to the plurality of ultrasonic element arrays have an overlapping region in which at least some of the measurement regions overlap each other in a plan view in the second direction.

Here, a transmission angle range in the ultrasonic element array can be set in advance according to measurement accuracy, an ultrasonic wave array structure, or the like.

In the application example with this configuration, the ultrasonic element array has a measurement region based on a preset transmission angle range. An overlapping region of measurement regions of the ultrasonic element arrays is disposed in the second direction. Therefore, it is possible to perform ultrasonic measurement at a plurality of measurement positions along the second direction.

In the ultrasonic device according to the application example, it is preferable that the transmission angle range is within ±45° with respect to a virtual line which is orthogonal to the first direction.

In the application example with this configuration, the transmission angle range in the ultrasonic element array is within ±45° with respect to the virtual line. Since a transmission angle in the ultrasonic element array is set to be within 45° as mentioned above, an ultrasonic wave transmitted from the ultrasonic element array can be caused to converge on a desired position, and an ultrasonic wave reflected from a measurement target can be appropriately received, so that highly accurate ultrasonic measurement can be performed.

In the ultrasonic device according to the application example, it is preferable that the ultrasonic element arrays are arranged in two or more columns and five or less columns in the first direction.

In the application example with this configuration, the ultrasonic element arrays are arranged in two or more columns and five or less columns in the first direction. In other words, two or more and five or less ultrasonic element arrays are arranged in the first direction. For example, in a configuration in which ultrasonic element arrays are arranged in two columns, an ultrasonic wave transmission angle can be secured in an equivalent manner to a case of the related art in which one column is arranged, and thus it is possible to reduce attenuation of an ultrasonic wave due to an increase in a transmission angle. In a case of five columns, it is possible to considerably reduce a measurement position interval. In a case of three or more columns and four or less columns, it is possible to reduce a measurement position interval while suppressing attenuation due to a transmission angle.

In the ultrasonic device according to the application example, it is preferable that in a case where a length dimension of the ultrasonic element array in the second direction is indicated by x, and an arrangement interval length of the plurality of ultrasonic element arrays in the second direction is indicated by Px, the ultrasonic element arrays included in n columns arranged in the first direction among the plurality of ultrasonic elements are disposed at positions satisfying (n−1)×Px≦x.

In the application example with this configuration, the ultrasonic device is configured so that the length dimension x of the ultrasonic element array in the second direction, the arrangement interval length Px in the second direction, and the arrangement number n of ultrasonic element arrays satisfy the relationship. Consequently, it is possible to perform ultrasonic measurement at n measurement positions within a range of the length dimension x in the second direction.

An ultrasonic measurement apparatus according to an application example includes any one of the ultrasonic devices of the application examples; and a control unit that controls the ultrasonic device.

The ultrasonic measurement apparatus of this application example includes any one of the ultrasonic devices of the application examples and the control unit controlling the ultrasonic device. In this configuration, it is possible to provide the ultrasonic measurement apparatus which can perform highly accurate ultrasonic measurement while reducing a measurement position interval in the same manner as in the application examples related to the ultrasonic devices.

In the ultrasonic measurement apparatus according to the application example, it is preferable that the control unit includes an angle setting portion that sets an ultrasonic wave transmission angle in the ultrasonic element array in a plane including the first direction.

In the application example with this configuration, the control unit includes the angle setting portion that sets an ultrasonic wave transmission angle in the ultrasonic element array. An ultrasonic wave transmission angle can be changed by the angle setting portion, and thus it is possible to change a position of a measurement region according to the ultrasonic wave transmission angle. For example, an ultrasonic wave transmission angle in each ultrasonic element array can be set so that a measurement target is disposed in a measurement region of the ultrasonic element array.

In the ultrasonic measurement apparatus according to the application example, it is preferable that the angle setting portion sets the transmission angle on the basis of a distance between a measurement position and the ultrasonic element array in a virtual line which is orthogonal to the first direction.

In the application example with this configuration, the angle setting portion sets a transmission angle on the basis of a distance between a measurement position and the ultrasonic element array. For example, in a case where a distance to a measurement position is short, a transmission angle in the ultrasonic element array is increased, and, in a case where the distance is long, a transmission angle is reduced. Therefore, it is possible to appropriately set a measurement region in each ultrasonic element array according to the distance. Consequently, even in a case where a distance between the ultrasonic element array and a measurement target is changed, a measurement position can be set to a position of a measurement target, and thus it is possible to appropriately perform ultrasonic measurement.

An ultrasonic image display according to an application example includes any one of the ultrasonic measurement apparatuses of the application examples; and an image display device.

In this application example, the ultrasonic image display includes any one of the ultrasonic measurement apparatuses of the application examples, and an image display device. In this configuration, it is possible to perform highly accurate ultrasonic measurement while reducing a measurement position interval, and thus to display an ultrasonic image based on a measurement result, in the same manner as in the application examples related to the ultrasonic devices. Therefore, it is possible to display a highly accurate ultrasonic image acquired with a narrow interval, and thus to improve visibility to an observer.

### BRIEF DESCRIPTION OF THE DRAWINGS

The invention will be described with reference to the accompanying drawings, wherein like numbers reference like elements.

FIG. 1 is a perspective view illustrating a schematic configuration of an ultrasonic measurement apparatus according to a first embodiment.

FIG. 2 is a block diagram illustrating a schematic configuration of the ultrasonic measurement apparatus according to the first embodiment.

FIG. 3 is a plan view illustrating a schematic configuration of an element board of an ultrasonic device in the first embodiment.

FIG. 4 is a sectional view of the ultrasonic device taken along the line A-A in FIG. 3.

FIG. 5 is a plan view schematically illustrating arrangement of an ultrasonic element array according to the first embodiment.

FIG. 6 is a side view schematically illustrating arrangement of the ultrasonic element array according to the first embodiment.

FIG. 7 is a flowchart illustrating an example of an ultrasonic measurement process according to the first embodiment.

FIG. 8 is a diagram schematically illustrating the ultrasonic element array according to the first embodiment.

FIG. 9 is a diagram schematically illustrating the ultrasonic element array according to the first embodiment.

FIG. 10 is a diagram schematically illustrating an ultrasonic element array according to a second embodiment.

FIG. 11 is a plan view schematically illustrating an ultrasonic element array according to a third embodiment.

FIG. 12 is a diagram schematically illustrating the ultrasonic element array according to the third embodiment.

FIG. 13 is a plan view schematically illustrating an ultrasonic element array according to a modification example.

### DESCRIPTION OF EXEMPLARY EMBODIMENTS

### First Embodiment

Hereinafter, an ultrasonic measurement apparatus according to a first embodiment will be described with reference to the drawings.

### Configuration of Ultrasonic Measurement Apparatus

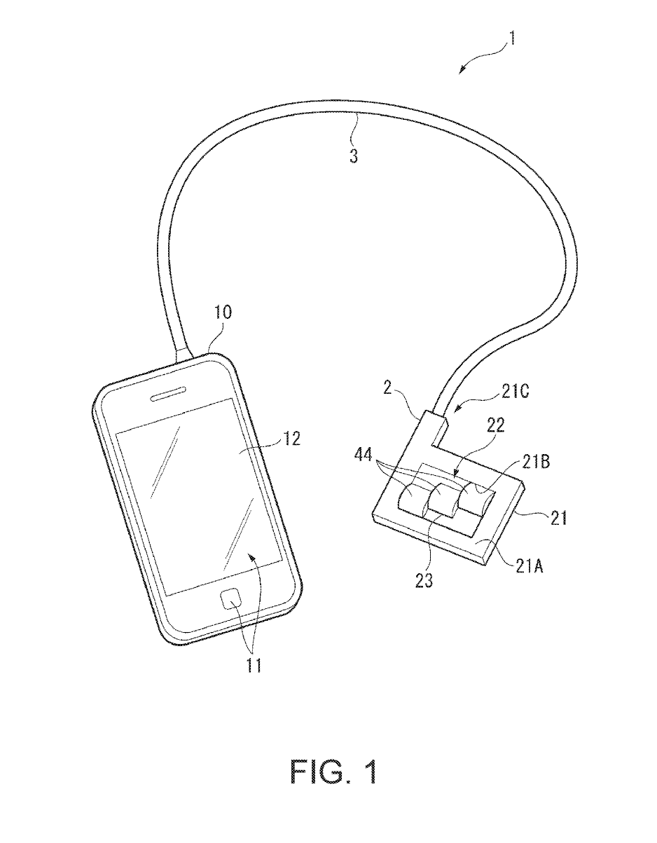

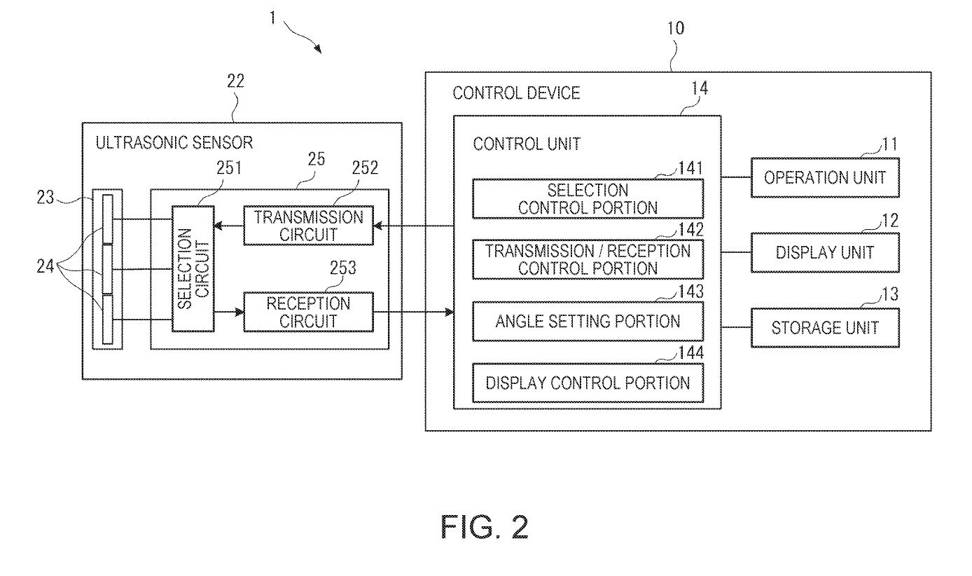

FIG. 1 is a perspective view illustrating a schematic configuration of an ultrasonic measurement apparatus 1 according to the present embodiment. FIG. 2 is a block diagram illustrating a schematic configuration of the ultrasonic measurement apparatus 1.

The ultrasonic measurement apparatus 1 of the present embodiment corresponds to an ultrasonic measurement apparatus and an ultrasonic image display, and, as illustrated in FIG. 1, includes an ultrasonic probe 2 and a control device 10 connected to the ultrasonic probe 2 via a cable 3.

The ultrasonic measurement apparatus 1 sends ultrasonic waves into a living body from the ultrasonic probe 2 in a state in which the ultrasonic probe 2 is brought into contact with a surface of the living body (human body). Ultrasonic waves reflected from an organ in the living body are received by the ultrasonic probe 2, and, for example, an internal tomographic image of the living body is obtained or a state (for example, a blood flow) of an organ in the living body is measured, on the basis of a received signal.

Configuration of Ultrasonic Probe

The ultrasonic probe 2 includes a casing 21 and an ultrasonic sensor 22 accommodated in the casing 21. The ultrasonic sensor 22 includes an ultrasonic device 23 provided with a plurality of ultrasonic element arrays 24 transmitting and receiving ultrasonic waves, and a circuit board 25 (refer to FIG. 2) provided with a driver circuit and the like controlling the ultrasonic device 23.

### Configuration of Casing

As illustrated in FIG. 1, the casing 21 is formed in a rectangular box shape in a plan view, and is provided with a sensor window 21B on one surface (sensor surface 21A) which is orthogonal to a thickness direction, and a part of the ultrasonic device 23 is exposed to the one surface. A passing hole 21C of the cable 3 is provided at a part (a side surface in the example illustrated in FIG. 1) of the casing 21. The cable 3 is inserted into the casing 21 through the passing hole 21C so as to be connected to the circuit board 25. A gap between the cable 3 and the passing hole 21C is filled with, for example, a resin material, and thus water resistance is ensured.

In the present embodiment, a configuration example in which the ultrasonic probe 2 is connected to the control device 10 via the cable 3 is described, but this is only an example, and, for example, the ultrasonic probe 2 and the control device 10 may be connected to each other via wireless communication, and various constituent elements of the control device 10 may be provided in the ultrasonic probe 2.

### Configuration of Ultrasonic Device

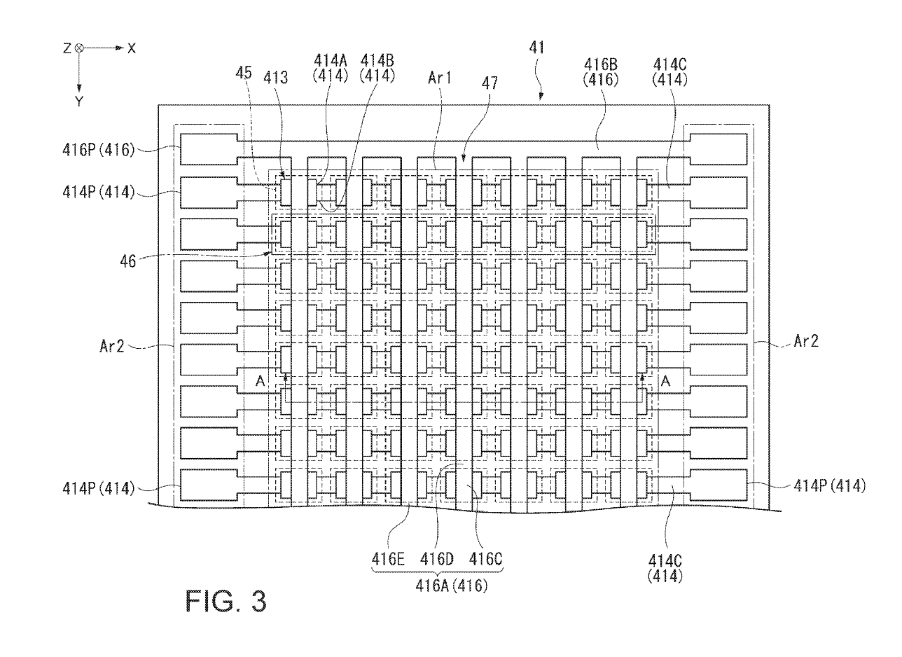

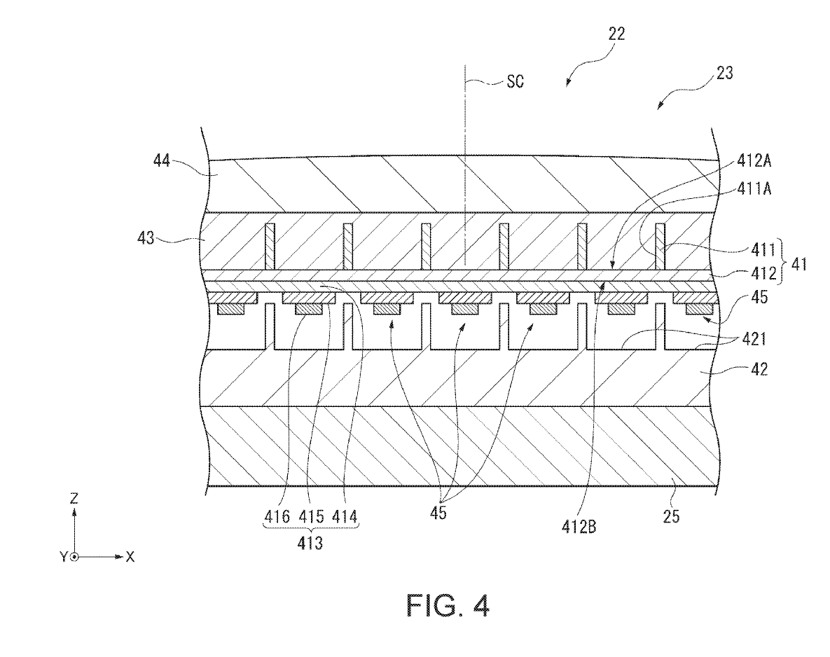

FIG. 3 is a plan view in which an element board 41 of the ultrasonic device 23 is viewed from a sealing plate 42 side. FIG. 4 is a sectional view of the ultrasonic device 23 taken along the line A-A in FIG. 3.

The ultrasonic device 23 includes a plurality of ultrasonic element arrays 24. The plurality of ultrasonic element arrays 24 are disposed on the circuit board 25 with a predetermined positional relationship. Each of the ultrasonic element arrays 24 is configured to be able to be driven separately.

### Configuration of Ultrasonic Element Array

As illustrated in FIG. 4, each of the ultrasonic element array 24 is formed of an element board 41, a sealing plate 42, an acoustic matching layer 43, and an acoustic lens 44.

The ultrasonic element array 24 includes a one-dimensional ultrasonic array 47 in which a plurality of ultrasonic wave transmission/reception portions 46 each of which functions as a single transmission/reception channel transmitting and receiving ultrasonic waves are disposed along a Y direction (which is a scan direction and corresponds to a first direction according to the invention). In the present embodiment, the ultrasonic element array 24 includes a plurality of ultrasonic transducers (ultrasonic elements) 45 disposed in a matrix form along an X direction and the Y direction, and, among the ultrasonic transducers, the plurality of ultrasonic transducers 45 disposed along the X direction (which is a slice direction and corresponds to a second direction according to the invention) form the ultrasonic wave transmission/reception portion 46 which functions as a single transmission/reception channel. The ultrasonic wave transmission/reception portions 46 can be driven separately, and transmit ultrasonic waves toward a +Z side. Since the ultrasonic wave transmission/reception portions 46 can be driven separately, the ultrasonic element array 24 can apply an ultrasonic wave beam along a virtual plane (hereinafter, also referred to as a scanning plane SC) passing through the center of the one-dimensional ultrasonic array 47 in the X direction in parallel to a YZ plane. The scanning plane SC is a plane including a central line which passes through the center of the ultrasonic element array 24 (the one-dimensional ultrasonic array 47 in the present embodiment) in the X direction and is parallel to the Y direction.

Hereinafter, a description will be made of a configuration of the ultrasonic element array 24.

### Configuration of Element Board

As illustrated in FIG. 4, the element board 41 includes a board main body portion 411, a vibration film 412 provided on the sealing plate 42 side of the board main body portion 411, and a piezoelectric element 413 provided on the vibration film 412. Here, in the following description, a surface (first surface) of the vibration film 412 opposite to the sealing plate 42 will be referred to as an ultrasonic wave transmission/reception surface 412A, and a surface (second surface) thereof on the sealing plate 42 side will be referred to as an operation surface 412B. In a plan view in which the element board 41 is viewed from a board thickness direction, a central region of the element board 41 is an array region Ar1, and the plurality of ultrasonic transducers 45 are disposed in a matrix form.

The board main body portion 411 is a board supporting the vibration film 412, and is formed of, for example, a semiconductor substrate such as Si. An opening 411A corresponding to each of the ultrasonic transducers 45 is provided in the array region Ar1 of the board main body portion 411. Each opening 411A is closed by the vibration film 412 provided on a surface of the board main body portion 411 on the sealing plate 42 side.

The vibration film 412 is made of, for example, SiO₂ or a laminate of SiO₂ and ZrO₂, and is provided to entirely cover the board main body portion 411 on the sealing plate 42 side. A thickness dimension of the vibration film 412 is sufficiently smaller than that of the board main body portion 411. In a case where the board main body portion 411 is made of Si, and the vibration film 412 is made of SiO₂, for example, the board main body portion 411 is subject to an oxidation process, and thus the vibration film 412 with a desired thickness dimension can be easily formed. In this case, the board main body portion 411 is subject to an etching process by using the vibration film 412 of SiO₂ as an etching stopper, and thus it is possible to easily form the opening 411A.

As illustrated in FIG. 4, the piezoelectric element 413 which is a laminate of a lower electrode 414, a piezoelectric film 415, and an upper electrode 416 is provided on the operation surface 412B of the vibration film 412 closing each opening 411A. Here, a single ultrasonic transducer 45 is formed of the vibration film 412 closing the opening 411A and the piezoelectric element 413.

In the ultrasonic transducer 45, a rectangular wave voltage with a predetermined frequency is applied between the lower electrode 414 and the upper electrode 416 so that the vibration film 412 in an opening region of the opening 411A vibrates, and thus an ultrasonic wave can be sent from the ultrasonic wave transmission/reception surface 412A side. If the vibration film 412 vibrates due to an ultrasonic wave which is reflected from a target object and is incident from the ultrasonic wave transmission/reception surface 412A side, a potential difference occurs between an upper part and a lower part of the piezoelectric film 415. Therefore, the received ultrasonic wave can be detected by detecting the potential difference occurring between the lower electrode 414 and the upper electrode 416.

In the present embodiment, as illustrated in FIG. 3, the plurality of ultrasonic transducers 45 are disposed along the X direction (slice direction) and the Y direction (scan direction) intersecting (in the present embodiment, orthogonal to) the X direction in the predetermined array region Ar1 of the element board 41.

The lower electrodes 414 of the ultrasonic transducers 45 arranged in the X direction are connected to each other, so as to form a single ultrasonic wave transmission/reception portion 46. In other words, in the ultrasonic wave transmission/reception portion 46, the lower electrodes 414 are provided to cross the plurality of ultrasonic transducers 45 arranged along the X direction, and are formed in a linear shape along the X direction. Each of the lower electrodes 414 is formed of a lower electrode main body 414A located between the piezoelectric film 415 and the vibration film 412, a lower electrode line 414B connecting adjacent lower electrode main bodies 414A to each other, and a lower terminal electrode line 414C extracted to each of terminal regions Ar2 other than the array region Ar1. Therefore, in the ultrasonic transducers 45 arranged in the X direction, the lower electrodes 414 have the same potential.

The lower terminal electrode line 414C extends to the terminal region Ar2 other than the array region Ar1 so as to form a first electrode pad 414P in the terminal region Ar2. The first electrode pad 414P is connected to a terminal portion provided on the wiring board.

On the other hand, as illustrated in FIG. 3, the upper electrode 416 includes element electrode portions 416A provided to cross the plurality of ultrasonic transducers 45 along the Y direction, and a common electrode portion 416B connecting ends of the plurality of element electrode portions 416A to each other. Each of the element electrode portions 416A includes an upper electrode main body 416C laminated on the piezoelectric film 415, an upper electrode line 416D connecting adjacent upper electrode main bodies 416C to each other, and an upper terminal electrode 416E extending outward along the Y direction from the ultrasonic transducers 45 which are disposed at both ends in the Y direction.

The common electrode portion 416B is provided at each of a +Y side end and a −Y side end of the array region Ar1. The common electrode portion 416B on the +Y side connects the upper terminal electrodes 416E to each other which extend toward the +Y side from the ultrasonic transducers 45 provided at the +Y side end among the plurality of ultrasonic transducers 45 provided along the Y direction. The common electrode portion 416B at the −Y side end connects the upper terminal electrodes 416E extending toward the −Y side to each other. Therefore, in the respective ultrasonic transducers 45 in the array region Ar1, the upper electrodes 416 have the same potential. The pair of common electrode portions 416B are provided along the X direction, and ends thereof are extracted to the terminal regions Ar2 from the array region Ar1. The common electrode portions 416B form second electrode pads 416P connected to the terminal portions of the wiring board in the terminal regions Ar2.

In the above-described ultrasonic element array 24, each ultrasonic wave transmission/reception portion 46 can be driven separately, and functions as a single transmission/reception channel. The plurality of ultrasonic wave transmission/reception portions 46 are disposed along the Y direction so as to form the one-dimensional ultrasonic array 47. The ultrasonic element array 24 can change a transmission angle of an ultrasonic wave along the scanning plane SC since each ultrasonic wave transmission/reception portion 46 is separately driven.

### Configuration of Sealing Plate

A planar shape of the sealing plate 42 viewed from the thickness direction is formed to be the same as, for example, that of the element board 41, and is formed of a semiconductor substrate such as Si or an insulator substrate. A material or a thickness of the sealing plate 42 influences frequency characteristics of the ultrasonic transducer 45, and is thus preferably set on the basis of a center frequency of an ultrasonic wave which is transmitted and received in the ultrasonic transducer 45.

The sealing plate 42 is provided with a plurality of grooves 421 corresponding to the openings 411A of the element board 41 in an array opposing region which opposes the array region Ar1 of the element board 41. Consequently, a gap with a predetermined dimension is provided between the vibration film 412 and the element board 41 in a region (inside the opening 411A) which is subject to vibration due to the ultrasonic transducer 45, and thus vibration of the vibration film 412 is not hindered. It is possible to prevent a problem (crosstalk) that a back wave from a single ultrasonic transducer 45 is incident to another ultrasonic transducer 45 adjacent thereto.

If the vibration film 412 vibrates, an ultrasonic wave as a back wave is emitted not only to the opening 411A side (ultrasonic wave transmission/reception surface 412A side) but also to the sealing plate 42 side (operation surface 412B side). The back wave is reflected by the sealing plate 42, and is emitted to the vibration film 412 side again via the gap. In this case, if phases of the reflected back wave and the ultrasonic wave emitted to the ultrasonic wave transmission/reception surface 412A side from the vibration film 412 are deviated relative to each other, the ultrasonic wave attenuates. Therefore, in the present embodiment, a depth of each of the grooves 421 is set so that an acoustic distance in the gap is an odd-numbered multiple of X/4 when a wavelength of the ultrasonic wave is indicated by X. In other words, a thickness dimension of each portion of the element board 41 or the sealing plate 42 is set by taking into consideration the wavelength X of an ultrasonic wave emitted from the ultrasonic transducer 45.

The sealing plate 42 may have a configuration in which openings (not illustrated) are provided to correspond to the electrode pads 414P and 416P provided in the terminal regions Ar2 at positions of the element board 41 opposing the terminal regions Ar2. In this case, through electrodes (through-silicon via (TSV)) which penetrate through the sealing plate 42 in the thickness direction are provided in the opening, and thus the electrode pads 414P and 416P are connected to the terminal portions of the wiring board via the through electrodes. There may be a configuration in which flexible printed circuits (FPC), cables, or wires are inserted into the openings so that the electrode pads 414P and 416P are connected to the wiring board.

### Configuration of Acoustic Matching Layer and Acoustic Lens

As illustrated in FIG. 4, the acoustic matching layer 43 is provided on the element board 41 on an opposite side to the sealing plate 42. Specifically, the acoustic matching layer 43 fills between the element board 41 and the acoustic lens 44, and is formed with a predetermined thickness dimension from the surface of the board main body portion 411.

The acoustic lenses 44 are provided on the acoustic matching layer 43, and are exposed to the outside of the sensor window 21B of the casing 21 as illustrated in FIG. 1. The acoustic lens 44 has a cylindrical shape as a result of a surface thereof on the +Z side being curved along the X direction (slice direction), and causes an ultrasonic wave transmitted from each ultrasonic wave transmission/reception portion 46 of the ultrasonic element array 24 to converge along the scanning plane SC.

The acoustic matching layer 43 or the acoustic lens 44 causes an ultrasonic wave transmitted from the ultrasonic transducer 45 to propagate toward a living body which is a measurement target with high efficiency, and causes an ultrasonic wave reflected inside the living body to propagate toward the ultrasonic transducer 45 with high efficiency. Thus, each the acoustic matching layer 43 and the acoustic lens 44 is set to acoustic impedance similar to acoustic impedance of a living body. As a material having such acoustic impedance, for example, silicon may be used.

### Arrangement of Ultrasonic Element Arrays

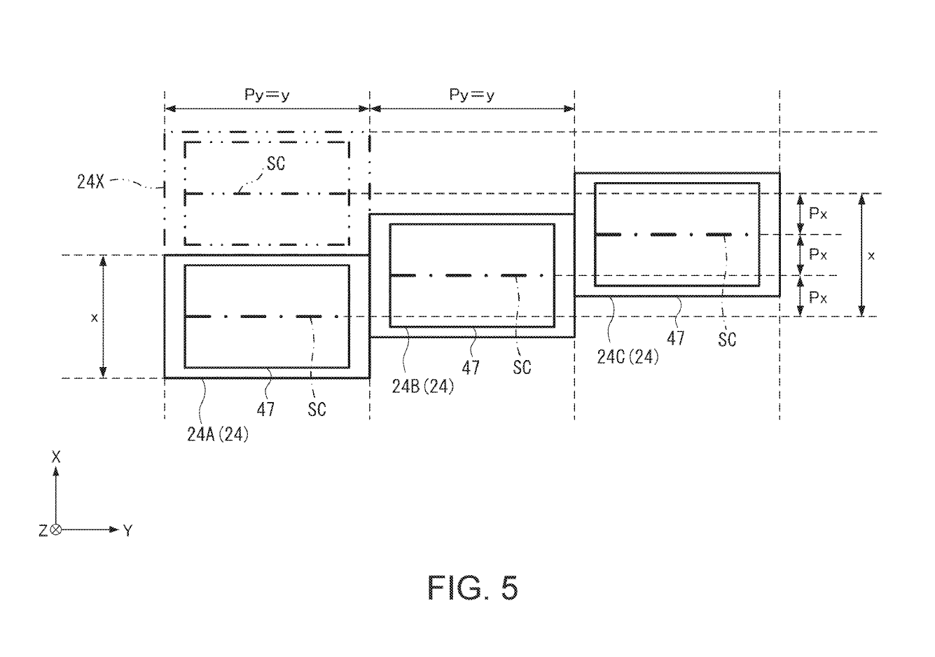

FIG. 5 is a plan view schematically illustrating arrangement of the plurality of ultrasonic element arrays 24 in a case where the ultrasonic device 23 is viewed from the −Z side.

As illustrated in FIG. 5, the ultrasonic device 23 includes the ultrasonic element arrays 24 of odd-numbered columns (in the present embodiment, three), and the three ultrasonic element arrays 24 are disposed in the Y direction. Hereinafter, for description, the three ultrasonic element arrays 24 are respectively referred to as a first ultrasonic element array 24A, a second ultrasonic element array 24B, and a third ultrasonic element array 24C from the −Y side.

In the present embodiment, the respective ultrasonic element arrays 24A, 24B and 24C are disposed at positions which are adjacent to each other in the Y direction (scan direction) and do not interfere with each other, and positions which are deviated in the X direction.

In other words, when an outer dimension of each of the ultrasonic element arrays 24A, 24B and 24C in the Y direction is referred to as a first dimension y, an arrangement interval length Py of each of the ultrasonic element arrays 24A, 24B and 24C in the Y direction is the same as the first dimension y. Here, a deviation amount of each scanning plane SC (that is, the central line including the scanning plane SC) in the Y direction is the arrangement interval length Py.

When an outer dimension of each of the ultrasonic element arrays 24A, 24B and 24C in the X direction is referred to as a second dimension x, an arrangement interval length Px of each of the ultrasonic element arrays 24A, 24B and 24C in the X direction is smaller than the second dimension x. In FIG. 5, Px is x/3. In other words, the scanning plane SC of each of the ultrasonic element arrays 24A, 24B and 24C is disposed with the arrangement interval length Px smaller than the second dimension x in the X direction (slice direction). In other words, arrangement positions of the ultrasonic element arrays 24 adjacent to each other in the Y direction partially overlap each other in the X direction.

The interval length Px between the scanning planes SC in the present embodiment configured in the above-described way is smaller than an interval (second dimension x) between the scanning planes SC in a case where the ultrasonic element array 24 such as a ultrasonic element array 24X indicated by two-dot chain lines in FIG. 5 is disposed to be adjacent along the X direction.

Here, in the ultrasonic device 23 of the present embodiment, the second dimension x, the arrangement interval length Px in the X direction, and the number n (in the present embodiment, n=3) of ultrasonic element arrays 24 arranged in the Y direction satisfy a relationship of the following Expression (1). In other words, in the present embodiment, a plurality of scanning planes SC are disposed at a predetermined interval (arrangement interval length Px) shorter than the second dimension x of the ultrasonic element array 24.

(n−1)×Px≦x (1)

### Measurement Region of Ultrasonic Element Array

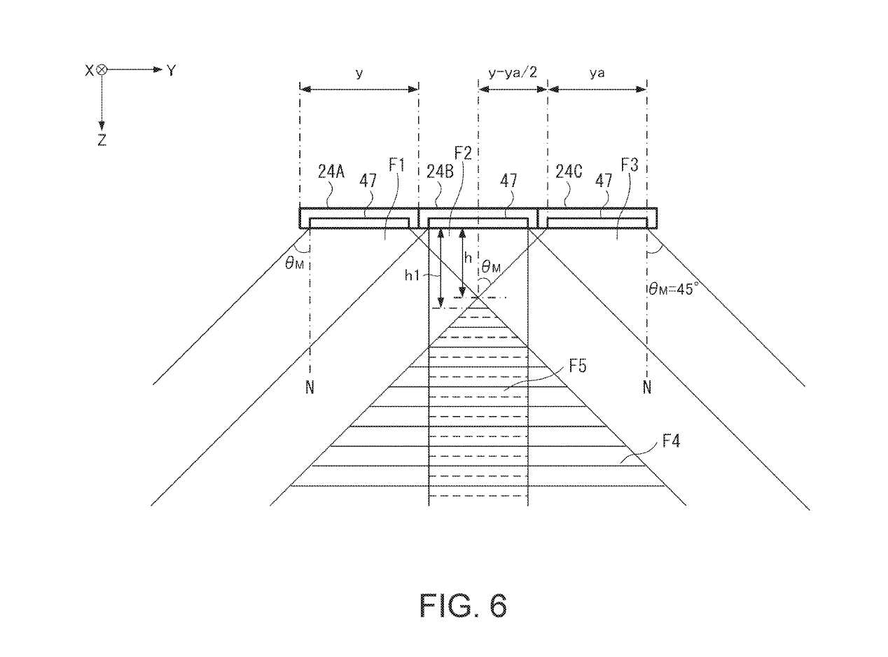

FIG. 6 is a side view in which the plurality of ultrasonic element arrays 24 illustrated in FIG. 5 are viewed from the −X side.

In the present embodiment, as illustrated in FIG. 6, each of the ultrasonic element arrays 24A, 24B and 24C applies an ultrasonic wave beam within an angle range of ±θ_{M} (for example, 45°) with respect to a normal line N to the one-dimensional ultrasonic array 47 under the control of the control device 10. The normal line N is a virtual line which is orthogonal to the X direction and the Y direction. The angle range is a set value of an ultrasonic wave transmission angle range when the ultrasonic element array 24 is driven under the control of a control unit which will be described later, and is a value which is equal to or less than the maximum value of an angle at which the ultrasonic element array 24 can transmit an ultrasonic wave.

In this case, the ultrasonic element arrays 24A, 24B and 24C can respectively perform ultrasonic measurement on measurement regions F1, F2 and F3. The measurement regions F1, F2 and F3 have a first overlapping region F4 in which the regions overlap each other in the X direction. If a measurement target is disposed at a position overlapping the first overlapping region F4 in the X direction, the ultrasonic element arrays 24A, 24B and 24C can perform ultrasonic measurement.

As will be described later, in the present embodiment, ultrasonic measurement is performed by disposing a measurement target at a position where an ultrasonic wave transmission angle of the second ultrasonic element array 24B is 0°. In this case, a region in which the measurement regions F1, F2 and F3 overlap each other in the X direction is a second overlapping region F5 illustrated in FIG. 6. In the second overlapping region F5, an ultrasonic wave is transmitted from the second ultrasonic element array 24B at a transmission angle of 0°, that is, in the Z direction. Thus, it is possible to improve measurement accuracy in ultrasonic measurement using the second ultrasonic element array 24B.

Here, the minimum distance h between the first overlapping region F4 and the second overlapping region F5, and the ultrasonic element arrays 24 in the Z direction is expressed by the following Equation (2). A dimension ya is a dimension of the one-dimensional ultrasonic array 47 in the Y direction. The number n of arranged ultrasonic element arrays 24 (hereinafter, also referred to as the arrangement number n) is 2K+1 (where K is an integer of 1 or more, and K=1 in the present embodiment). The first dimension y of the ultrasonic element array 24, the dimension ya of the one-dimensional ultrasonic array 47, and the transmission angle θ_{M} are set so that the distance h is equal to or less than a target value h1 of a measurement distance. Consequently, a measurement target in which a measurement distance is equal to or more than the target value h1 can be disposed in the first overlapping region F4 and the second overlapping region F5.

h=(Ky−ya/2)×cot θ_{M} (2)

### Configuration of Circuit Board

The circuit board 25 is provided with a driver circuit and the like for controlling the ultrasonic transducers 45, and is bonded to the ultrasonic device 23 via, for example, a wiring member such as a flexible printed circuit (FPC) (not illustrated). The circuit board 25 is provided with, as illustrated in FIG. 2, a selection circuit 251, a transmission circuit 252, and a reception circuit 253.

The selection circuit 251 is connected to the ultrasonic device 23, and switches between transmission connection for connecting any one of the ultrasonic element arrays 24 to the transmission circuit 252 and reception connection for connecting any one thereof to the reception circuit 253 under the control of the control device 10.

The transmission circuit 252 outputs a transmission signal for transmitting an ultrasonic wave, to any one of the ultrasonic element arrays 24 of the ultrasonic device 23 via the selection circuit 251 when switching to the transmission connection occurs under the control of the control device 10.

The reception circuit 253 outputs a received signal which is input from the ultrasonic device 23 via the selection circuit 251 to the control device 10 when switching to the reception connection occurs under the control of the control device 10. The reception circuit 253 is configured to include, for example, a low noise amplification circuit, a voltage controlled attenuator, a programmable gain amplifier, a low-pass filter, and an A/D converter, and performs signal processes such as conversion of the received signal to a digital signal, removal of a noise component, and amplification to a desired signal level, and outputs the received signal having undergone the processes to the control device 10.

### Configuration of Control Device

As illustrated in FIG. 2, the control device 10 is configured to include, for example, an operation unit 11, a display unit 12, a storage unit 13, and a control unit 14. As the control device 10, for example, a terminal device such as a tablet terminal, a smart phone, or a personal computer may be used, and a dedicated terminal device for operating the ultrasonic probe 2 may be used. The control device 10 corresponds to an image display device according to the invention.

The operation unit 11 is a user interface (UI) for a user operating the ultrasonic measurement apparatus 1, and may be formed of, for example, a touch panel provided on the display unit 12, an operation button, a keyboard, or a mouse.

The display unit 12 is formed of, for example, a liquid crystal display, and displays an image.

The storage unit 13 stores various programs and various pieces of data for controlling the ultrasonic measurement apparatus 1.

The control unit 14 is formed of, for example, a calculation circuit such as a central processing unit (CPU), and a storage circuit such as a memory. The control unit 14 reads the various programs stored in the storage unit 13 and executes the programs, so as to function as a selection control portion 141, a transmission/reception control portion 142, an angle setting portion 143, and a display control portion 144, and controls the ultrasonic device 23 and the display unit 12.

The selection control portion 141 controls the selection circuit 251 to select a single ultrasonic element array 24 performing transmission and reception of ultrasonic waves from among the plurality of ultrasonic element arrays 24, and performs switching to the transmission connection during transmission of an ultrasonic wave and switching to the reception connection during reception of an ultrasonic wave. If transmission and reception of ultrasonic waves in the single ultrasonic element array 24 are completed, the selection control portion 141 changes the ultrasonic element array 24 performing transmission and reception of ultrasonic waves. In the present embodiment, for example, the selection control portion 141 selects the ultrasonic element arrays 24A, 24B and 24C in this order.

The transmission/reception control portion 142 controls transmission and reception of ultrasonic waves. The transmission/reception control portion 142 controls, for example, the selection circuit 251 so as to connect the ultrasonic device 23 to the transmission circuit 252 during transmission of an ultrasonic wave, and to connect the ultrasonic device 23 to the reception circuit 253 during reception of an ultrasonic wave. The transmission/reception control portion 142 controls the transmission circuit 252 to perform a process of generating and outputting a transmission signal, and controls the reception circuit 253 to perform a process of setting a frequency of a received signal or setting a gain thereof.

The transmission/reception control portion 142 controls ultrasonic wave transmission angles of the respective ultrasonic element arrays 24A, 24B and 24C on the basis of set values of ultrasonic wave transmission angles set by the angle setting portion 143.

The angle setting portion 143 sets ultrasonic wave transmission angles of the respective ultrasonic element arrays 24A, 24B and 24C. The angle setting portion 143 sets transmission angles in the respective ultrasonic element arrays 24A, 24B and 24C on the basis of, for example, distances between the respective ultrasonic element arrays 24A, 24B and 24C and a measurement position in the Z direction.

In the present embodiment, as will be described later, the angle setting portion 143 sets transmission angles in the first ultrasonic element array 24A and the third ultrasonic element array 24C according to a distance from the center (the center of the second ultrasonic element array 24B) of the ultrasonic device 23 in the Y direction.

The display control portion 144 generates an ultrasonic image (for example, a B mode image) on the basis of ultrasonic measurement results in the respective ultrasonic element arrays 24A, 24B and 24C. The display control portion 144 displays the generated ultrasonic image on the display unit 12.

### Ultrasonic Measurement Process in Ultrasonic Measurement Apparatus

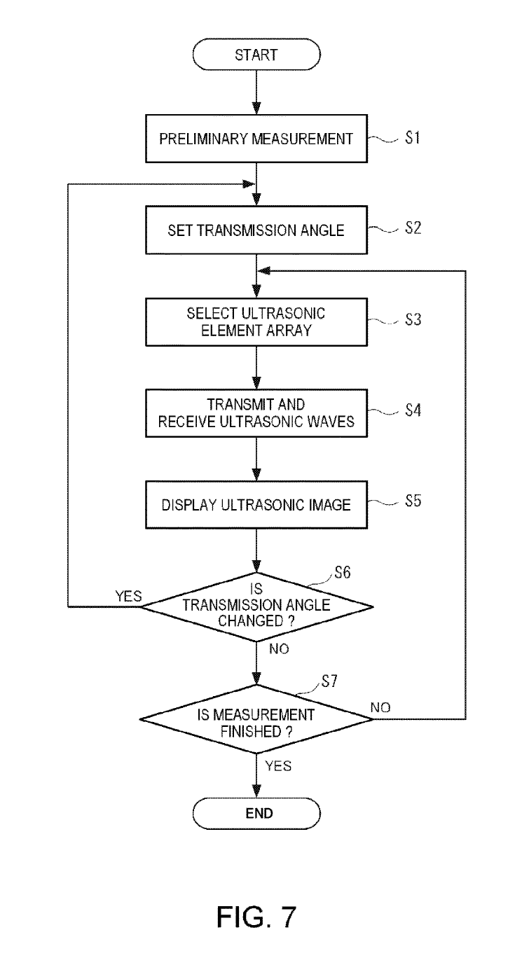

FIG. 7 is a flowchart illustrating an example of an ultrasonic measurement process in the ultrasonic measurement apparatus 1.

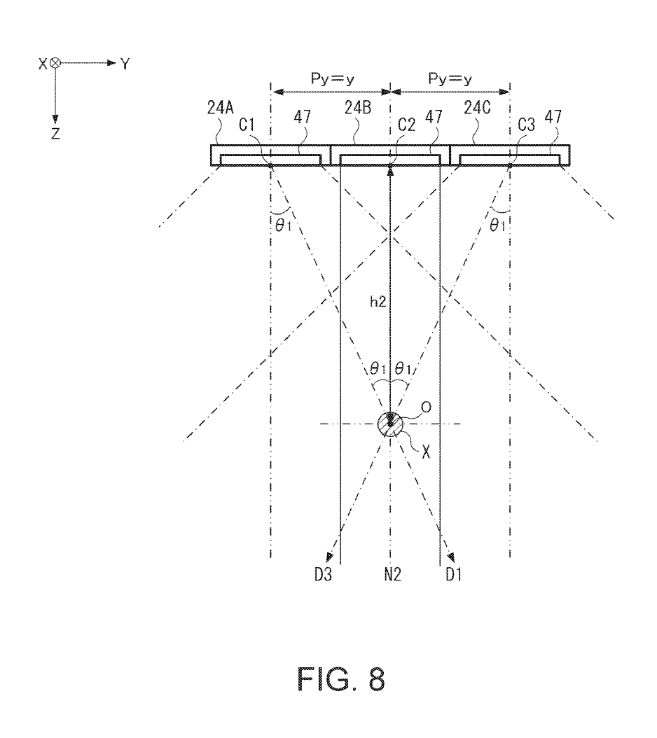

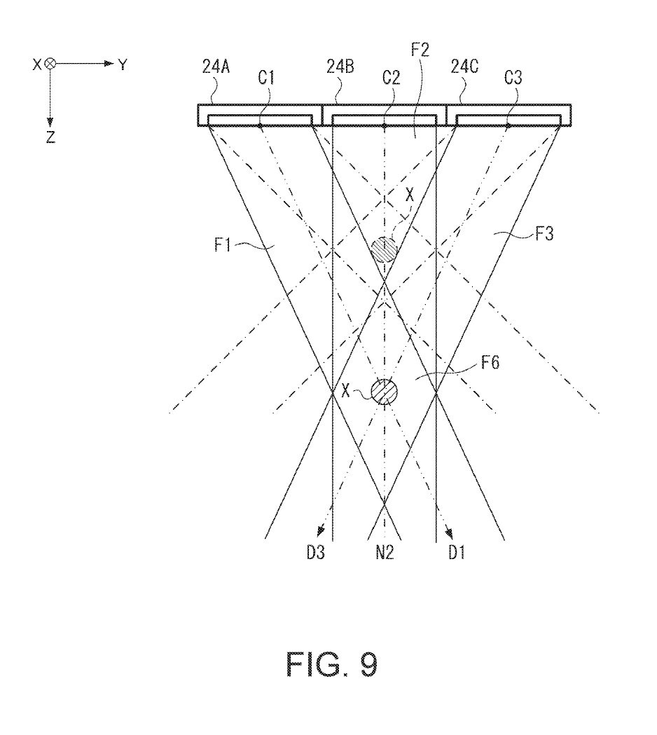

FIGS. 8 and 9 are diagrams schematically illustrating the ultrasonic element arrays 24 in the ultrasonic measurement process illustrated in FIG. 7.

Hereinafter, a description will be made of the ultrasonic measurement process in the ultrasonic measurement apparatus 1. The ultrasonic measurement apparatus 1 of the present embodiment may be used for, for example, puncture work of inserting a puncture needle into a predetermined organ (for example, a blood vessel) in a living body. In other words, when an operator inserts the puncture needle into the living body in the X direction, the operator can more easily recognize a position of the puncture needle by checking (observing) an ultrasonic image which is generated on the basis of ultrasonic measurement results in the plurality of ultrasonic element arrays 24 and is displayed on the display unit 12.

As illustrated in FIG. 7, first, the ultrasonic measurement apparatus 1 performs preliminary measurement for setting a transmission angle in each ultrasonic element array (step S1).

In step S1, the selection control portion 141 controls the selection circuit 251 to connect either of the transmission circuit 252 and the reception circuit 253 to the second ultrasonic element array 24B located at the center in the Y direction among the plurality of ultrasonic element arrays 24. The transmission/reception control portion 142 drives the second ultrasonic element array 24B to transmit and receive ultrasonic waves. At this time, as illustrated in FIG. 8, the ultrasonic probe 2 is fixed to a body surface so that the second ultrasonic element array 24B is located directly over (−Z side) a measurement target X (observation position). A position where the ultrasonic probe 2 is fixed is adjusted, for example, by the operator referring to the ultrasonic image acquired during the preliminary measurement.

Next, as illustrated in FIG. 8, the angle setting portion 143 sets transmission angles in the first ultrasonic element array 24A and the third ultrasonic element array 24C on the basis of a distance h2 in the Z direction between the measurement target X and the ultrasonic element arrays 24 (step S2).

Here, in FIG. 8, ultrasonic wave transmission directions of the first ultrasonic element array 24A and the third ultrasonic element array 24C are indicated by D1 and D3, and the magnitude of the transmission angle is indicated by θ1. In FIG. 8, when projected onto the YZ plane (a plane parallel to each scanning plane), a point O (indicating a central position of the measurement target X in the illustrated example) is set at the distance h2 in a normal line N2 to the second ultrasonic element array 24B, passing through a center C2 of the one-dimensional ultrasonic array 47.

The angle setting portion 143 sets the transmission direction D1 in the first ultrasonic element array 24A as a vector which is directed from a center C1 of the first ultrasonic element array 24A toward the point O. The angle setting portion 143 sets the transmission angle θ1 in the first ultrasonic element array 24A as θ1 satisfying the following Equation (3). In the present embodiment, since Py is y, θ1 satisfying the following Equation (4) is set.

The angle setting portion 143 sets the transmission direction D3 and the transmission angle θ1 in the third ultrasonic element array 24C as follows. In other words, the transmission direction D3 is set as a vector which is directed from a center C3 of the third ultrasonic element array 24C toward the point O, and the transmission angle θ1 is set to satisfy the following Equations (3) and (4).

tan θ1=Py/h2 (3)

tan θ1=y/h2 (4)

As mentioned above, in the present embodiment, the transmission angles and the transmission directions D1 and D3 in the ultrasonic element arrays 24A and 24C with the second ultrasonic element array 24B interposed therebetween along the Y direction are symmetric to each other with respect to the normal line N2 when projected onto the XY plane. In other words, the angle setting portion 143 sets the transmission angle θ1 on the basis of the distance h2 between the ultrasonic element arrays 24 and the measurement target X in the Z direction, and the distance (arrangement interval length Py) between the center C2 and the center of each of the ultrasonic element arrays 24 in the Y direction.

The distance h2 between the ultrasonic element arrays 24 and the measurement target X may be calculated on the basis of a coordinate of a measurement position designated by the operator. The coordinate of the measurement position is acquired, for example, by the operator designating the measurement position through an operation of the operation unit 11 while observing an ultrasonic image displayed on the display unit 12.

The control device 10 detects a position of a measurement target such as a blood vessel, for example, through edge detection or pattern detection in an ultrasonic image on the basis of a result of the preliminary measurement, and calculates and sets a transmission angle according to a detection result.

Next, the selection control portion 141 selects the ultrasonic element array 24 performing transmission and reception of ultrasonic waves (step S3). In the present embodiment, the first ultrasonic element array 24A, the second ultrasonic element array 24B, and the third ultrasonic element array 24C are driven in this order.

Next, the transmission/reception control portion 142 drives the ultrasonic element array 24 selected in step S3 to perform transmission and reception of ultrasonic waves (step S4). In step S4, first, the transmission/reception control portion 142 drives the ultrasonic element array 24 of a driving target to transmit an ultrasonic wave as illustrated in FIG. 9. Here, the transmission angle θ1 in each ultrasonic element array 24 is set to satisfy the above Equations (3) and (4), and the measurement target X is disposed at a substantially central position in an overlapping region F6 in which the respective measurement regions F1, F2 and F3 of the ultrasonic element arrays 24A, 24B and 24C overlap each other. The ultrasonic element array 24 as a driving target receives a reflected wave from the measurement target X. The reception circuit 253 performs various processes on a received signal from the ultrasonic element array 24, and outputs the received signal having undergone the processes to the control device 10.

Next, the display control portion 144 generates an ultrasonic image (for example, a B mode image) on the basis of an ultrasonic measurement result in the ultrasonic element array 24, and displays the generated ultrasonic image on the display unit 12 (step S5). In the present embodiment, a description has been made of an exemplary configuration in which an ultrasonic image is displayed, but the control device 10 may store a measurement result in the storage unit 13 instead of displaying the measurement result.

Next, the angle setting portion 143 determines whether or not the ultrasonic wave transmission angle is required to be changed (step S6).

For example, as illustrated in FIG. 9, if a position of the measurement target X is changed so as to be moved to the outside of the overlapping region F6, the measurement target X cannot be measured with the ultrasonic element arrays 24. In FIG. 9, the first and third ultrasonic element arrays 24A and 24C cannot measure the measurement target X, and thus it is necessary to reset the first and third ultrasonic element arrays 24A and 24C.

For example, in a case where the operator instructs an angle to be set by performing an input operation, the angle setting portion 143 determines YES in step S6. For example, in a case where a position of the measurement target X in the Z direction is changed by a predetermined value or greater (for example, 50% or more of a width of the overlapping region F6 in the Z direction) on the basis of a measurement result in the second ultrasonic element array 24B, the angle setting portion 143 determines YES in step S6. A width h3 of the overlapping region F6 in the Z direction may be roughly estimated by using, for example, the following Equation (5).

h3=ya×cot θ1 (5)

If YES is determined in step S6, the control unit 14 performs the processes in step S2 and the subsequent steps. As illustrated in FIG. 9, in step S2, the angle setting portion 143 resets transmission angles in the first and third ultrasonic element arrays 24A and 24C so that the moved measurement target X is included in the overlapping region.

On the other hand, if NO is determined in step S6, the control unit 14 determines whether or not an instruction for finishing the measurement is received (step S7). If NO is determined in step S7, the control unit 14 performs the processes in step S3 and the subsequent steps. If YES is determined, the control unit 14 finishes the ultrasonic measurement process.

### Operations and Effects of First Embodiment

In the plurality of ultrasonic element arrays 24, the ultrasonic wave transmission/reception portions 46 are arranged in the Y direction. The plurality of ultrasonic element arrays 24 do not overlap each other in the Y direction, and are disposed at positions which do not interfere with each other. In other words, a certain ultrasonic element array 24 and another ultrasonic element array 24 adjacent to the ultrasonic element array 24 in the Y direction are disposed to be adjacent to or separate from each other in the Y direction. The plurality of ultrasonic element arrays 24 are disposed at different positions in the X direction.

Each of the ultrasonic element arrays 24 has a predetermined outer dimension. Thus, in a case where the ultrasonic element arrays 24 are arranged in the X direction, an interval between the scanning planes SC are not smaller than a width dimension of the ultrasonic element array 24, and thus a measurement position interval is increased. In contrast, in the present embodiment, since the ultrasonic element arrays 24 are disposed at positions which do not overlap each other in the Y direction, the ultrasonic element arrays adjacent to each other in the X direction can be disposed at any positions. Therefore, a distance between the scanning planes SC, that is, a measurement position interval can be freely set. In other words, the ultrasonic element arrays 24 can be disposed at positions overlapping each other in the X direction, and thus a measurement position interval can be made smaller than an outer dimension of the ultrasonic element array 24.

Since the ultrasonic element arrays 24 are disposed in advance with a predetermined positional relationship, there is no influence of vibration or the like, and highly accurate ultrasonic measurement can be performed compared with a configuration in which the scanning plane SC is moved to any position by moving or rotating the ultrasonic element array 24.

As mentioned above, according to the present embodiment, it is possible to perform highly accurate ultrasonic measurement while reducing a measurement position interval. It is possible to display an ultrasonic image based on a measurement result. Therefore, it is possible to display a highly accurate ultrasonic image acquired with a narrow interval, and thus to improve visibility to an observer.

In the present embodiment, the ultrasonic element array 24 has a measurement region based on a preset transmission angle range. An overlapping region of measurement regions of the ultrasonic element arrays 24 is disposed in the X direction. Therefore, it is possible to perform ultrasonic measurement at a plurality of measurement positions along the X direction.

In the present embodiment, the length dimension x of the ultrasonic element array 24 in the X direction, the arrangement interval length Px in the X direction, and the arrangement number n of ultrasonic element arrays 24 satisfy a relationship of (n−1)×Px≦x. Consequently, it is possible to perform ultrasonic measurement at n measurement positions within a range of the length dimension x in the X direction.

In the present embodiment, a distance between the ultrasonic element array 24 and the overlapping region in a direction along the normal line N is equal to or less than the target value h1. In other words, the ultrasonic device 23 has an ultrasonic wave transmission angle range, an outer dimension of the ultrasonic element array 24, and a positional relationship between the plurality of ultrasonic element arrays 24 so that the distance is equal to or less than the preset target value h1. In this configuration, since a target value is set according to a distance between the ultrasonic element arrays 24 and the measurement target X, the measurement target X can be disposed in an overlapping region of the ultrasonic element arrays 24, and thus it is possible to perform ultrasonic measurement on the measurement target X at a plurality of measurement positions.

In the present embodiment, a transmission angle range in the ultrasonic element array 24 is within ±45°. Since a transmission angle in the ultrasonic element array 24 is set to be within 45° as mentioned above, an ultrasonic wave transmitted from the ultrasonic element array 24 can be caused to converge on a desired position, and an ultrasonic wave reflected from a measurement target can be appropriately received, so that highly accurate ultrasonic measurement can be performed.

In the present embodiment, the ultrasonic element arrays 24 in three columns are disposed in the Y direction. In this configuration, it is possible to prevent a distance between the ultrasonic element arrays 24 in the Y direction from being increasing, and thus to prevent an increase in an ultrasonic wave transmission angle. Therefore, it is possible to reduce a measurement position interval while suppressing attenuation of an ultrasonic wave due to an increase in a transmission angle. In the present embodiment, a transmission angle in the second ultrasonic element array 24B can be set to 0° by disposing a measurement target under the second ultrasonic element array 24B, and thus it is possible to prevent deterioration in the measurement accuracy of the second ultrasonic element array 24B. Ultrasonic wave transmission angles of other ultrasonic element arrays 24 adjacent to the second ultrasonic element array 24B are set to the same magnitude, and thus it is possible to prevent deterioration in the measurement accuracy of only one of the ultrasonic element arrays 24. Therefore, it is possible to more reliably reduce a measurement interval.

Here, in the present embodiment, an ultrasonic wave transmission angle can be changed by the angle setting portion 143, and thus it is possible to change a position of a measurement region according to the ultrasonic wave transmission angle. For example, an ultrasonic wave transmission angle in each ultrasonic element array can be set so that a measurement target is disposed in a measurement region of the ultrasonic element array 24.

Here, the angle setting portion 143 sets a transmission angle on the basis of the distance h2 between a measurement position and the ultrasonic element array 24. For example, in a case where the distance h2 is short, a transmission angle in the ultrasonic element array 24 is increased, and, in a case where the distance h2 is long, a transmission angle is reduced. Therefore, it is possible to appropriately set a measurement region in each ultrasonic element array 24 according to the distance. Consequently, even in a case where a distance between the ultrasonic element array 24 and a measurement target is changed, a measurement position can be set to a position of a measurement target, and thus it is possible to appropriately perform ultrasonic measurement.

### Second Embodiment

Next, a second embodiment will be described.

In the first embodiment, three ultrasonic element arrays 24 are configured to be disposed in the Y direction, that is, an odd number of ultrasonic element arrays 24 are configured to be disposed. In contrast, in the second embodiment, there is a difference in that an even number of, for example, two ultrasonic element arrays 24 are configured to be disposed.

Hereinafter, a reception transducer according to the present embodiment will be described. In the following description, the same constituent elements as in the first embodiment are given the same reference numerals, and description thereof will be omitted or made briefly.

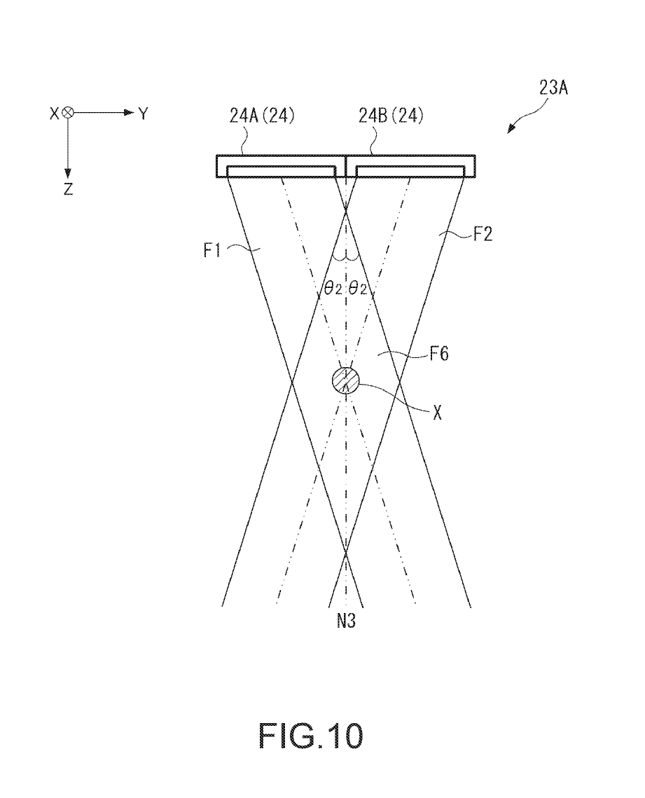

FIG. 10 is a diagram schematically illustrating each ultrasonic element array 24 of an ultrasonic device 23A according to the second embodiment.

The ultrasonic device 23A illustrated in FIG. 10 includes a first ultrasonic element array 24A and a second ultrasonic element array 24B. The ultrasonic device 23A is configured in the same manner as the ultrasonic device 23 of the first embodiment except that the third ultrasonic element array 24C is not provided.

In an ultrasonic measurement apparatus of the present embodiment, as illustrated in FIG. 10, the measurement target X is disposed on a virtual plane which passes between the first ultrasonic element array 24A and the second ultrasonic element array 24B in the Y direction and is parallel to the XZ plane. In other words, when projected onto the YZ plane (a plane which is parallel to each scanning plane), the measurement target X is disposed on a normal line N3 to each ultrasonic element array 24, passing between the first ultrasonic element array 24A and the second ultrasonic element array 24B in the Y direction.

Here, as in the present embodiment, in a case where an even number (that is, the arrangement number n=2K, and K=1 in the present embodiment) of ultrasonic element arrays 24 are provided, the minimum distance h between an overlapping region in which measurement regions of the respective ultrasonic element arrays 24 overlap each other and the ultrasonic element arrays 24 in the Z direction is expressed by the following Equation (6) in the same manner as in the first embodiment. Also in the present embodiment, the first dimension y of the ultrasonic element array 24, the dimension ya of the one-dimensional ultrasonic array 47, and the transmission angle θ_{M} are set so that the distance h is equal to or less than a target value of a measurement distance.

h={Ky−(y−ya)/2}×cot θ_{M} (6)

In an ultrasonic measurement process, in the same manner as in the first embodiment, the angle setting portion 143 sets a transmission angle θ2 in each of the first ultrasonic element array 24A and the second ultrasonic element array 24B to satisfy the following Equation (7) in the substantially same manner as in the transmission angle θ1 of the first embodiment.

Here, in the present embodiment, a distance between the first ultrasonic element array 24A and the normal line N3 is shorter than a distance between the first ultrasonic element array 24A and the normal line N2 in the first embodiment. Thus, in the second embodiment, under the condition in which a depth of a measurement target is the same as in the first embodiment, for example, a transmission angle may be smaller than a transmission angle in the first ultrasonic element array 24A of the first embodiment.

tan θ2=(Py/2)h2 (7)

Also in the ultrasonic measurement apparatus provided with the ultrasonic device 23A, the measurement target X is disposed directly under the second ultrasonic element array 24B, and thus an ultrasonic measurement process can be performed in the same manner as in the first embodiment. This is also the same for a case where the measurement target X is disposed directly under the first ultrasonic element array 24A.

### Operations and Effects of Second Embodiment

In the present embodiment, the ultrasonic element arrays 24 in two columns are disposed in the Y direction. In this configuration, it is possible to reduce a distance between the ultrasonic element arrays 24 in the Y direction, and thus to prevent an increase in an ultrasonic wave transmission angle. Therefore, it is possible to reduce a measurement position interval while suppressing attenuation of an ultrasonic wave due to an increase in a transmission angle.

Ultrasonic wave transmission angles of the two ultrasonic element arrays 24 are set to be the same as each other, and thus it is possible to prevent deterioration in the ultrasonic measurement accuracy of one of the ultrasonic element arrays 24. Consequently, quality of an ultrasonic image can be made uniform. The same image process is performed on two ultrasonic images which are acquired at the substantially same angle, and thus a process can be simplified.

### Third Embodiment

Next, a third embodiment will be described.

In the first embodiment, three ultrasonic element arrays 24 are configured to be disposed in the Y direction. In contrast, in the third embodiment, there is a difference in that an odd number of (five or more) ultrasonic element arrays 24 are configured to be disposed.

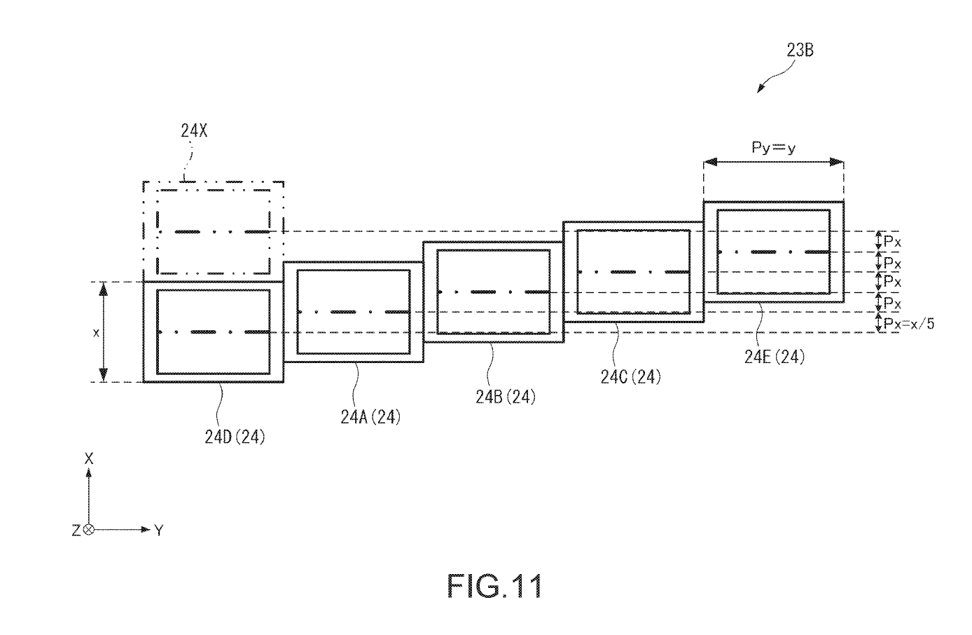

FIG. 11 is a plan view schematically illustrating ultrasonic element arrays 24 when an ultrasonic device 23B of the third embodiment is viewed from the −Z side.

The ultrasonic device 23B illustrated in FIG. 11 includes not only the first ultrasonic element array 24A, the second ultrasonic element array 24B, and the third ultrasonic element array 24C of the first embodiment, but also a fourth ultrasonic element array 24D and a fifth ultrasonic element array 24E.

Also in the present embodiment, the ultrasonic element arrays 24 are disposed to be adjacent to each other in the Y direction (scan direction) and are disposed with the arrangement interval length Px in the X direction. In the present embodiment, the arrangement interval length Px in the X direction is x/5.

Among the ultrasonic element arrays 24, the fourth ultrasonic element array 24D is disposed on the −Y side of the first ultrasonic element array 24A. An interval between the scanning planes SC in the X direction of the first ultrasonic element array 24A and the fourth ultrasonic element array 24D is Px.

The fifth ultrasonic element array 24E is disposed on the +Y side of the third ultrasonic element array 24C. An interval between the scanning planes SC in the X direction of the third ultrasonic element array 24C and the fifth ultrasonic element array 24E is Px.

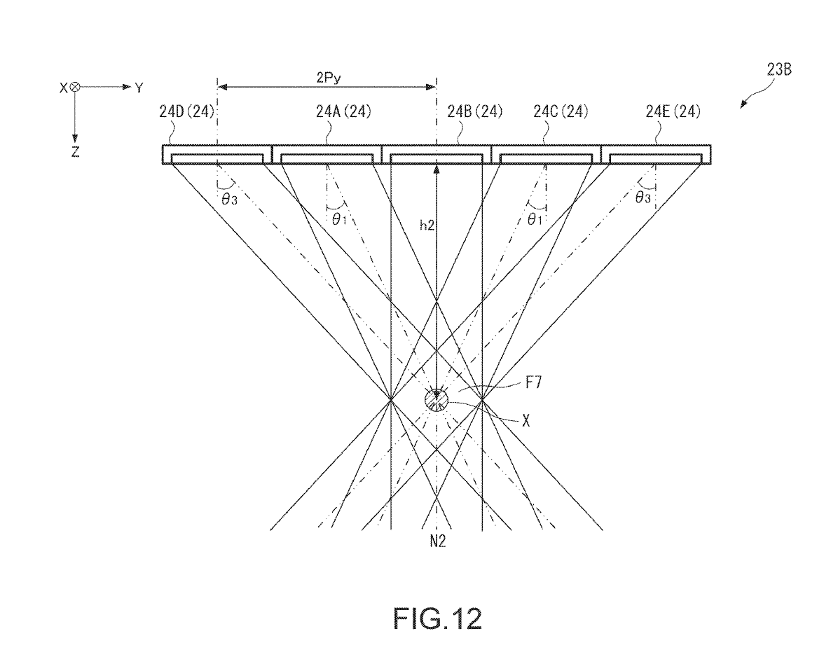

FIG. 12 is a side view in which the plurality of ultrasonic element arrays 24 illustrated in FIG. 11 are viewed from the −X side.

As illustrated in FIG. 12, the measurement target X is disposed in an overlapping region F7 in which measurement regions of the ultrasonic element arrays 24 overlap each other. As illustrated in FIG. 12, a transmission angle of each ultrasonic element array 24 is set. A transmission angle θ3 of each of the fourth ultrasonic element array 24D and the fifth ultrasonic element array 24E is set to θ3 satisfying the following Equation (8). The transmission angle θ3 is larger than the transmission angle θ2 of each of the first ultrasonic element array 24A and the third ultrasonic element array 24C.

tan θ3=2Py/h2 (8)

### Operations and Effects of Third Embodiment

In the present embodiment, the ultrasonic element arrays 24 in five columns are disposed in the Y direction. In this configuration, it is possible to considerably reduce a measurement position interval. In the present embodiment, since an odd number of ultrasonic element arrays 24 are disposed in the Y direction, a measurement target is disposed directly under the ultrasonic element array 24 disposed at the center, and thus an ultrasonic wave transmission angle of the ultrasonic element array 24 can be set to 0°. Since two sets of ultrasonic element arrays 24 are disposed with the ultrasonic element array 24 disposed at the center interposed therebetween, the ultrasonic element arrays 24 in each set are disposed at an equal distance from the center of the ultrasonic device 23. In other words, in each set, distances from the ultrasonic element arrays 24 to a measurement position are the substantially same as each other. Therefore, ultrasonic wave transmission angles in each set can be made the same as each other, and thus ultrasonic measurement can be performed with substantially equivalent accuracy.

### Modification Examples

The invention is not limited to the above-described respective embodiments, and configurations obtained through modifications, alterations, and combinations of the embodiments within the scope of being capable of achieving the object of the invention are included in the invention.

For example, in the above-described respective embodiments, two, three or five ultrasonic element arrays 24 are arranged in the Y direction, but the number of arranged ultrasonic element arrays 24 is not limited thereto, and any number of ultrasonic element arrays 24 may be disposed.

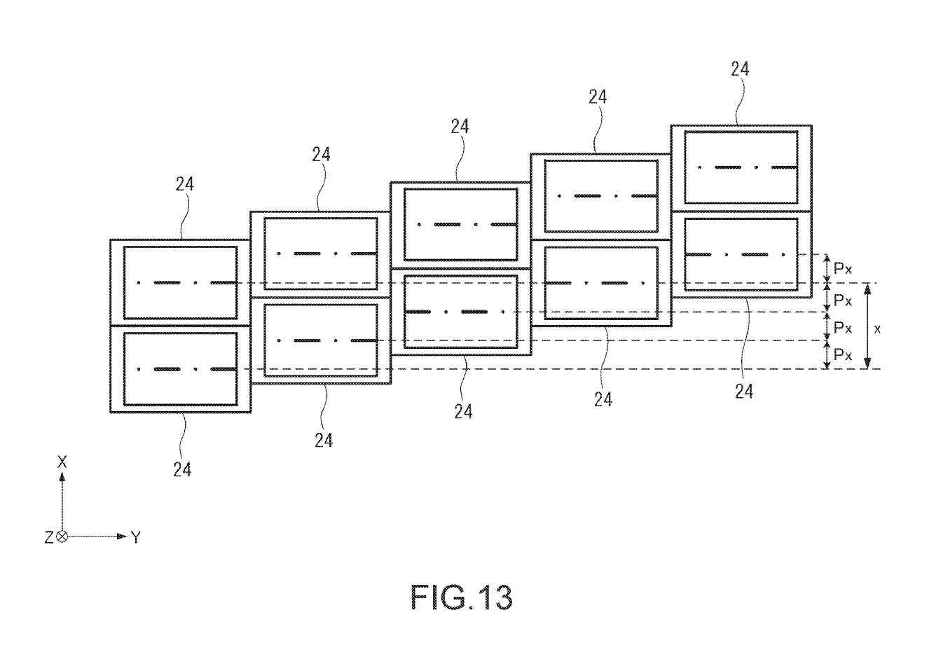

FIG. 13 is a diagram illustrating a modification example of arrangement of a plurality of ultrasonic element arrays 24.

In the above-described respective embodiments, a description has been made of an exemplary configuration in which plurality of ultrasonic element arrays 24 are disposed along the Y direction within a range smaller than the second dimension x of the ultrasonic element array 24 in the X direction, but any other configuration may be used. For example, as illustrated in FIG. 13, there may be a configuration in which columns of the ultrasonic element arrays 24 (hereinafter, also referred to as ultrasonic element array columns) disposed along the Y direction as described above are disposed in parallel to each other in the X direction (slice direction). The plurality of ultrasonic element arrays 24 may be disposed over a range larger than the second dimension x of the ultrasonic element array 24 in the X direction. Also in this configuration, a scanning plane interval can be reduced compared with a configuration in which the plurality of ultrasonic element arrays 24 are only disposed in the slice direction.

In other words, according to an aspect of the invention, among the plurality of ultrasonic element arrays, a first ultrasonic element array and a second ultrasonic element array overlapping the first ultrasonic element array when projected in a first direction are included. Consequently, compared with a configuration in which two ultrasonic element arrays are disposed in a second direction, a distance between scanning planes of the first ultrasonic element array and the second ultrasonic element array in the second direction can be reduced.

In the above-described respective embodiments, among the plurality of ultrasonic element arrays 24, ultrasonic element arrays 24 adjacent to each other in the Y direction are disposed to be in partial contact with each other, but this is only an example. For example, arrangement positions of ultrasonic element arrays 24 adjacent to each other in the Y direction may be separated from each other.

In the above-described respective embodiments, a description has been made of an exemplary configuration in which outer dimensions of the ultrasonic element arrays 24 are all the same as each other, but there may be a configuration in which ultrasonic element arrays with different dimensions may be combined with each other.

In the above-described respective embodiments, a description has been made of an exemplary configuration in which measurement regions of the plurality of ultrasonic element arrays 24 overlap each other in the X direction (slice direction), but any other configuration may be used. In other words, there may be a configuration in which some measurement regions of the plurality of ultrasonic element arrays 24 may overlap each other. All measurement regions of the ultrasonic element arrays 24 may not overlap each other in the X direction. For example, in a case where measurement regions overlap each other in a direction intersecting the X direction and the Y direction, a distance between scanning planes in the intersecting direction can be shorter than in a configuration of the related art.

In the above-described respective embodiments, a description has been made of an exemplary configuration in which an ultrasonic wave transmission angle of the ultrasonic element array 24 is within a range of ±45° with respect to a normal direction to the one-dimensional ultrasonic array 47, but this is only an example. The maximum value of an ultrasonic wave transmission angle may be more than 45°, and may be smaller than 45°. If the maximum value is 45°, an ultrasonic wave scanning range can be sufficiently secured compared with a case where the maximum value is less than 45°. If a transmission angle is equal to or less than 45°, it is possible to prevent a decrease in resolution due to being more than 45°.

In the above-described respective embodiments, the ultrasonic device 23 is configured so that a measurable distance in the Z direction is equal to or less than a preset target value, but this is only an example, and the ultrasonic device 23 may not be configured so that a measurable distance in the Z direction is equal to or less than a target value. In other words, there may be a configuration in which ultrasonic measurement is performed within a measurable distance range which is defined depending on a size of the ultrasonic element array 24 or a change range of an ultrasonic wave transmission angle without setting a target value.

In the above-described respective embodiments, a description has been made of an exemplary configuration in which the ultrasonic transducer 45 includes the vibration film 412 and the piezoelectric element 413 formed on the vibration film 412, but any other configuration may be used. For example, there may be a configuration in which the ultrasonic transducer 45 includes a vibration film and a vibrator (for example, an electrostatic actuator) causing the vibration film to vibrate.

In the embodiments, a description has been made of an exemplary configuration in which the ultrasonic measurement apparatus employs an organ of a living body as a measurement target, but the invention is not limited thereto. For example, the invention is applicable to a measuring machine which employs various structural bodies as measurement targets, and detects defects of the structural bodies or examines deterioration thereof. For example, the invention is applicable to a measuring machine which employs various semiconductor packages, wafers, or the like as measurement targets, and detects defects of the measurement targets.

A specific structure at the time of implementing the invention may be configured by combining the respective embodiments and modification examples with each other as appropriate within the scope of being capable of achieving the object of the invention, and may be altered to other structures as appropriate.

The entire disclosure of Japanese Patent Application No. 2016-065204 filed Mar. 29, 2016 is expressly incorporated by reference herein.

What is claimed is:

1. An ultrasonic device comprising:
a plurality of ultrasonic element arrays each of which is provided with a plurality of ultrasonic elements arranged in a first direction,
wherein the plurality of ultrasonic element arrays are disposed at positions which do not interfere with each other in the first direction, and are disposed at positions which are deviated relative to each other in a second direction intersecting the first direction.

2. The ultrasonic device according to claim 1,
wherein a deviation amount in the second direction in ultrasonic element arrays adjacent to each other in the first direction among the plurality of ultrasonic element arrays is smaller than a width in the second direction of each of the ultrasonic element arrays adjacent to each other.

3. The ultrasonic device according to claim 1,
wherein each of the ultrasonic element arrays has a measurement region based on a set transmission angle range of an ultrasonic wave in a plane including the first direction, and
wherein the measurement regions corresponding to the plurality of ultrasonic element arrays have an overlapping region in which at least some of the measurement regions overlap each other in a plan view in the second direction.

4. The ultrasonic device according to claim 3,
wherein the transmission angle range is within ±45° with respect to a virtual line which is orthogonal to the first direction.

5. The ultrasonic device according to claim 1,
wherein the ultrasonic element arrays are arranged in two or more columns and five or less columns in the first direction.

6. The ultrasonic device according to claim 1,
wherein, in a case where a length dimension of the ultrasonic element array in the second direction is indicated by x, and an arrangement interval length of the plurality of ultrasonic element arrays in the second direction is indicated by Px, the ultrasonic element arrays included in n columns arranged in the first direction among the plurality of ultrasonic elements are disposed at positions satisfying (n−1)×Px≦x.

7. An ultrasonic measurement apparatus comprising:
the ultrasonic device according to claim 1; and
a control unit that controls the ultrasonic device.

8. An ultrasonic measurement apparatus comprising:
the ultrasonic device according to claim 2; and
a control unit that controls the ultrasonic device.

9. An ultrasonic measurement apparatus comprising:
the ultrasonic device according to claim 3; and
a control unit that controls the ultrasonic device.

10. An ultrasonic measurement apparatus comprising:
the ultrasonic device according to claim 4; and
a control unit that controls the ultrasonic device.

11. An ultrasonic measurement apparatus comprising:
the ultrasonic device according to claim 5; and
a control unit that controls the ultrasonic device.

12. An ultrasonic measurement apparatus comprising:
the ultrasonic device according to claim 6; and
a control unit that controls the ultrasonic device.

13. The ultrasonic measurement apparatus according to claim 7,
wherein the control unit includes an angle setting portion that sets an ultrasonic wave transmission angle in the ultrasonic element array in a plane including the first direction.

14. The ultrasonic measurement apparatus according to claim 13,
wherein the angle setting portion sets the transmission angle on the basis of a distance between a measurement position and the ultrasonic element array in a virtual line which is orthogonal to the first direction.

15. An ultrasonic image display comprising:
the ultrasonic measurement apparatus according to claim 7; and
an image display device.

16. An ultrasonic image display comprising:
the ultrasonic measurement apparatus according to claim 8; and
an image display device.

17. An ultrasonic image display comprising:
the ultrasonic measurement apparatus according to claim 9; and
an image display device.

18. An ultrasonic image display comprising:
the ultrasonic measurement apparatus according to claim 10; and
an image display device.

19. An ultrasonic image display comprising:
the ultrasonic measurement apparatus according to claim 13; and
an image display device.

20. An ultrasonic image display comprising:
the ultrasonic measurement apparatus according to claim 14; and
an image display device.
